(19) United States
(12) Patent Application Publication
(10) Pub. No.: US 2026/0224774 A1
(43) Pub. Date: Aug. 6, 2026

(54) USE OF IN SITU SOLIDIFYING INJECTABLE COMPOSITIONS FOR FILLING A VOID IN A SUBJECT

(71) Applicant: FLUIDX MEDICAL TECHNOLOGY, INC., Salt Lake City, UT (US)

(72) Inventors: Russell J. Stewart, Salt Lake City, UT (US); Karen Ginster, Salt Lake City, UT (US); Daniel K. Smith, Salt Lake City, UT (US)

(21) Appl. No.: 19/576,014

(22) Filed: Mar. 24, 2026

Related U.S. Application Data
(63) Continuation-in-part of application No. PCT/US2024/048061, filed on Sep. 24, 2024.
(60) Provisional application No. 63/585,062, filed on Sep. 25, 2023.

(51) Int. Cl. A61L 27/18

(52) U.S. Cl. CPC A61L 27/18; A61L 2430/22

(57) ABSTRACT

Described herein are injectable compositions that have properties suited for filling a void in a subject. The injectable compositions are composed of (i) one or more polycationic polyelectrolytes and anionic counterions and (ii) one or more one polyanionic polyelectrolytes and cationic counterions. The injectable compositions have an ion concentration that is sufficient to prevent association of the polycationic polyelectrolytes and the polyanionic polyelectrolytes in water. For example, the counterions are of sufficient concentration to prevent the polycations and polyanions from associating electrostatically, which results in the formation of a stable injectable composition. Upon introduction of the composition into a subject, a solid is produced in situ. The viscosity of the injectable compositions can be varied depending upon the application of the injectable composition. By varying the molecular weight, charge densities, and/or concentrations of the polycationic and polyanionic salts, it is possible to produce injectable compositions having a useful range of viscosities.

### CROSS-REFERENCE TO RELATED APPLICATIONS

This application is a continuation in-part of International Application No. PCT/US2024/048061, filed on Sep. 24, 2024, which claims the benefit of and priority to co-pending U.S. Provisional Patent Application No. 63/585,062, filed on Sep. 25, 2023, the contents of which are incorporated by reference herein in their entireties.

### BACKGROUND

The left atrial appendage (LAA) is a structure in the cardiac anatomy with important pathological associations. First, thrombus has a predilection to form within the LAA in patients with non-valvar atrial fibrillation and to a lesser extent in those with mitral valve disease (both in atrial fibrillation and in sinus rhythm). Second, the use of transoesophageal echocardiography has made clear imaging of the LAA possible, so that its size, shape, flow patterns, and content can be assessed in health and disease.

The risk of stroke is increased approximately fivefold in non-rheumatic atrial fibrillation and 17-fold in patients with mitral stenosis and atrial fibrillation. About 15% of ischaemic strokes arise as a result of atrial fibrillation. Approximately 90% of atrial thrombi in non-rheumatic atrial fibrillation and 60% of such thrombi in patients with rheumatic mitral valve disease (predominantly stenosis) are seen within the LAA.

Although the incidence of thromboembolism in atrial fibrillation and mitral valve disease can be dramatically reduced with the use of anticoagulants, particularly warfarin, the use of such treatment can be complicated, and is contraindicated in many patients. Alternative forms of treatment are needed for the prophylaxis of thromboembolism in these patients.

### SUMMARY

Described herein are injectable compositions that have properties suited for filling a void in a subject. The injectable compositions are composed of (i) one or more polycationic polyelectrolytes and anionic counterions and (ii) one or more one polyanionic polyelectrolytes and cationic counterions. The injectable compositions have an ion concentration that is sufficient to prevent association of the polycationic polyelectrolytes and the polyanionic polyelectrolytes in water. For example, the counterions are of sufficient concentration to prevent the polycations and polyanions from associating electrostatically, which results in the formation of a stable injectable composition. Upon introduction of the composition into a subject, a solid is produced in situ. The viscosity of the injectable compositions can be varied depending upon the application of the injectable composition. By varying the molecular weight, charge densities, and/or concentrations of the polycationic and polyanionic salts, it is possible to produce injectable compositions having a useful range of viscosities.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the filling the left atrial appendage with the injectable composition described herein.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims.

The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

In describing and claiming the disclosed subject matter, the following terminology will be used in accordance with the definitions set forth below.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a solvent” includes, but are not limited to, mixtures or combinations of two or more such solvents, and the like.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed and “about 5 to about 15” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions disclosed herein as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

A weight percent of a component, unless specifically stated to the contrary, is based on the total weight of the formulation or composition in which the component is included. Weight percent includes and covers weight/volume percent and weight/weight percent.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and the like. Examples of longer chain alkyl groups include, but are not limited to, a palmitate group. A “lower alkyl” group is an alkyl group containing from one to six carbon atoms.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc.

The term “treat” as used herein is defined as maintaining or reducing the symptoms of a pre-existing condition when compared to the same symptoms in the absence of the injectable composition. The term “prevent” as used herein is the ability of the injectable compositions described herein to completely eliminate the activity or reduce the activity when compared to the same activity in the absence of the injectable composition. The term “inhibit” as used herein refers to the ability of the injectable composition to slow down or prevent a process.

As used herein, the term “introducing” with respect to administering the composition described herein in a void present in a subject includes a mode of administration that is oral, topical, intravenous, subcutaneous, percutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by catheters, stents or via an implanted reservoir or other device that administers, either actively or passively (e.g. by diffusion) a composition the perivascular space and adventitia. The term “parenteral” can include subcutaneous, percutaneous, intravenous, intramuscular, intra-articular, intra-synovial, intrasternal, intrathecal, intrahepatic, intralesional, and intracranial injections or infusion techniques.

“Subject” refers to mammals including, but not limited to, humans, non-human primates, sheep, dogs, rodents (e.g., mouse, rat, guinea pig, etc.), cats, rabbits, cows, horses, and non-mammals including vertebrates, birds, fish, amphibians, and reptiles.

The term “salt” as used herein is defined as a dry solid form of a water-soluble compound that possesses cations and anions. When the salt is added to water, the salt dissociates into cations and anions. A polycationic salt is a compound having a plurality of cationic groups with anionic counterions. A polyanionic salt is a compound having a plurality of anionic groups with cationic counterions.

The term “polyelectrolytes” as used herein is defined as polymers with ionized functional groups, where the ionized functional groups can incorporated in the polymer backbone, a sidechain of the polymer, or a combination thereof. Polycations and polyanions are produced when a polycationic salt or a polyanionic salt is dissolved in water.

The term “molecular weight” is used herein to refer to the average molecular mass of an ensemble of synthetic polymers that contains a distribution of molecular masses. Unless otherwise noted, values reported herein are weight-average molecular weight (Mw).

The term “stable solution” as used herein is defined as a liquid composition of oppositely charged polyelectrolytes that do not interact electrostatically. The polyelectrolyte solutions do not separate into macroscopically distinct phases.

The term “solid” as used herein is defined as a non-fluid, viscoelastic material that has a substantially higher elastic modulus and viscous modulus than the initial fluid form of the injectable composition used to produce the solid.

The term “transient” as used herein with respect to the contrast agent is defined herein as the ability of the contrast agent to diffuse or escape over time the solid produced by the injectable compositions described herein.

The term “temporary contrast” as used herein occurs when the majority of the transient contrast agent diffuses from the solid such that the transient contrast agent cannot be detected in the subject by imaging techniques such as, for example, fluoroscopy or CT.

The term “critical ion concentration” is the concentration of ions above which a specific combination of polycations and polyanions do not associate electrostatically, thus preventing liquid-liquid or liquid-sold phase separation. The critical ion concentration for a specific composition depends on multiple factors, including the molecular weight and concentration of the polyelectrolyte pairs, the mol % of polymeric ions, the polymeric ion species, the free ion species, and pH. The counterions that dissociate from the polymeric salts upon dissolution in water contribute to the total ion concentration of the solution. In most cases, for the polyelectrolyte pairs and concentrations described herein, the concentration of dissociated counterions is above the critical ion concentration for the specific composition. In some cases, additional ions (e.g., monovalent ions such as NaCl) can be added to increase the total ion concentration to above the critical ion concentration for the specific composition.

“Physiological conditions” refers to conditions such as osmolality, ion concentrations, pH, temperature, etc. within a particular area of the subject. For example, the normal blood sodium concentration range is between 135 and 145 mMol/L in a human.

As used herein, a plurality (i.e., more than one) of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an example, a numerical range of “about 1” to “about 5” should be interpreted to include not only the explicitly recited values of about 1 to about 5, but also to include individual values and sub-ranges within the indicated range. Thus, included in this numerical range are individual values such as 2, 3, and 4, the sub-ranges such as from 1-3, from 2-4, from 3-5, from about 1-about 3, from 1 to about 3, from about 1 to 3, etc., as well as 1, 2, 3, 4, and 5, individually. The same principle applies to ranges reciting only one numerical value as a minimum or maximum. Furthermore, such an interpretation should apply regardless of the breadth or range of the characters being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference of each various individual and collective combination and permutation of these compounds may not be explicitly disclosed, each is specifically contemplated and described herein. For example, if a class of molecules A, B, and C are disclosed, as well as a class of molecules D, E, and F, and an example of a combination A+D is disclosed, then even if each is not individually recited, each is individually and collectively contemplated. Thus, in this example, each of the combinations A+E, A+F, B+D, B+E, B+F, C+D, C+E, and C+F, are specifically contemplated and should be considered disclosed from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered disclosed from disclosure of A, B, and C; D, E, and F; and the example combination of A+D. This concept applies to all aspects of this disclosure including, but not limited to, steps in methods of making and using the disclosed compositions. Thus, if there exist a variety of additional steps that can be performed with any specific embodiment or combination of embodiments of the disclosed methods, each such combination is specifically contemplated and should be considered disclosed.

### Injectable In Situ Solidifying Injectable Compositions

Described herein are injectable compositions produced by mixing at least one polycationic salt, and at least one polyanionic salt. Upon addition to water, the polycationic salt and polyanionic salt dissociate to produce a solution of polycations, polyanions, and counterions. The concentration of the counterions in solution is greater than the critical ion concentration of the composition, which is sufficient to prevent electrostatic association and subsequent separation of the polyelectrolytes into distinct liquid or solid phases. The application site within a subject has total ion concentrations below the ion concentration of the injectable composition, resulting in polyelectrolyte association and formation of a solid upon administration of the injectable composition into the subject.

Upon introduction of the injectable composition into the subject (e.g., within a blood vessel), the counterions present in the injectable composition diffuse out from the composition. Diffusion of ions out of the injectable composition allows electrostatic interactions between polycations and polyanions present in the composition, resulting in conversion of the polyelectrolytes into a non-fluid, water-insoluble solid in situ. The solid produced in situ is a stiff cohesive material that remains positioned at the site of solidification within the subject.

One advantage of the injectable compositions is the viscosity of the composition can be modified or fine-tuned depending upon the application of the injectable composition. As will be discussed in detail below, varying parameters such as, for example, the concentration and/or molecular weight of the polycationic salt and polyanionic salt can be used to modify the viscosity of the composition. Furthermore, the concentration of the transient contrast agent can also be used to modify the viscosity of the composition. This makes the injectable compositions versatile in a number of different applications, as the injectable compositions can be introduced (i.e., administered) to the void in the subject using needles, catheters, microcatheters or other delivery devices having a wide range of internal diameters and lengths that require the use of injectable compositions having different viscosities.

Another design criteria of liquid embolics is the viscosity of the composition. Viscosity determines the size of microcatheter through which an embolic can be delivered. A key factor in the ability to deliver a liquid embolic is the burst pressure of the microcatheter, the highest hydrodynamic pressure it can withstand as the fluid is pushed through the catheter. This pressure is determined and specified for each commercial microcatheter. These burst pressures vary from 300 psi to 1200 psi, but 800 psi is a common value for high-end embolic microcatheters. A variety of factors influence the maximum hydrodynamic pressure on the catheter. These factors are related by Poiseuille's equation where P is pressure, r is the radius of the tube (catheter), L is the length of the catheter, Q is the volumetric flow rate, and p is viscosity of the embolic. This equation assumes a steady laminar flow through a cylindrical tube, which are generally appropriate for this application.

Poiseulle's equation: ΔP=8QμL/πr⁴

This equation predicts maximum hydrodynamic pressure within the catheter, assuming pressure at the end of the catheter is at or reasonably close to zero and Newtonian fluid behavior. As a result, the burst pressure of the catheter constrains properties of the embolic agent, catheter, and delivery rate. The most consequential of these factors is the internal radius of the catheter since it is related to pressure by the inverse fourth power, meaning that decreasing the catheter radius by half increases the hydrodynamic pressure 16-fold. While careful selection of catheter size is important for successful embolization, it is in many ways limited by the specific application. For example, many situations require directing catheters into blood vessels less than 1 mm in diameter, necessitating the use of catheters <3 F (1 mm) in outer diameter. These catheters have internal diameters no greater than 0.027″ (0.69 mm). Some highly selective or neurovascular applications require catheters less than 2 F in outer diameter, which have internal diameters less than 0.014″ (0.36 mm). Given the limitations of controlling catheter diameter, control of other parameters is required to ensure successful application. Other factors within the equation that are directly proportional to hydrodynamic pressure are catheter length, flow rate of the material, and viscosity of the material. Length of the catheter and flow rate of material are properties are also largely governed by the procedure specifics or operator preference, leaving viscosity of the material as the primary factor for controlling injectability.

Finally, the injectable compositions can be readily and easily prepared as needed. As will be discussed below, the injectable compositions can be prepared in a number of different ways depending upon the application of the compositions.

Each component used to produce the injectable compositions described herein as well as methods for making the injectable compositions is provided below.

### Polycationic Salts

The polycationic salt is compound having a plurality of cationic groups and pharmaceutically-acceptable counterions, where there is a 1:1 stoichiometric ratio of the cationic groups to anionic counterions. In one aspect, the polycationic salt is a polymer having a polymer backbone with a plurality of cationic groups and pharmaceutically-acceptable anionic counterions. The cationic groups can be pendant to the polymer backbone and/or incorporated within the polymer backbone.

In one aspect, the polycationic polyelectrolyte is derived by dissolving a polycationic salt in water. In one aspect, the polycationic salt is a polycationic hydrochloride salt, wherein upon mixing with water produces the polycationic polyelectrolyte and chloride ions. In another aspect, the polycationic salts described herein can be produced by combining a polymer with a plurality of basic groups (e.g., amino groups) with an acid to produce the corresponding cationic groups. In various aspects, acids which may be employed to form pharmaceutically acceptable polycationic salts include inorganic acids as hydrochloric acid, acetic acid, or other monovalent carboxylic acids.

Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

In other aspects, when the polycationic salt is a polymer, the polycationic salt can be produced by the polymerization of one or more monomers, where the monomers possess one or more cationic groups with corresponding counterion. Non-limiting procedures for making the polycationic salts using this approach are provided in the Examples. In one aspect, once the polycation has been prepared, excess ions can be removed from the polycation by filtration or dialysis prior to drying (e.g., lyophilization) to produce the polycationic salt with stoichiometric amounts of anionic counterions relative to the number of cationic groups.

In one aspect, the counterion of the polycationic salt is a monovalent ion such as, for example, chloride, pyruvate, acetate, tosylate, benzenesulfonate, benzoate, lactate, salicylate, glucuronate, galacturonate, nitrite, mesylate, trifluoroacetate, nitrate, gluconate, glycolate, formate, or any combination thereof. In one aspect, the counterion of the polycationic salt is a multivalent ion such as, for example, sulfate or phosphate.

In one aspect, the polycationic salt is a pharmaceutically-acceptable salt of a polyamine. The amino groups of the polyamine can be branched or part of the polymer backbone. In one aspect, the polyamine comprises two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.

In one aspect, the pharmaceutically-acceptable salt of the polyamine can include an aryl group having one or more amino groups directly or indirectly attached to the aromatic group. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine. In another aspect, the biodegradable polyamine can be gelatin modified with ethylenediamine.

The amino group of the polyamine can be protonated at a pH of from about 6 to about 9 (e.g., physiological pH) to produce cationic ammonium groups with a pharmaceutically-acceptable counterion.

In general, the polyamine salt is a polymer with a large excess of positive charges relative to negative charge at or near physiological pH. For example, the polycationic salt can have from 10 to 90 mole %, 10 to 80 mole %, 10 to 70 mole %, 10 to 60 mole %, 10 to 50 mole %, 10 to 40 mole %, 10 to 30 mole %, or 10 to 20 mole % protonated amino groups. In another aspect, all of the amino groups of the polyamine are protonated.

In one aspect, the polycationic salt can have a protonated residue of lysine, histidine, or arginine. For example, arginine has a guanidinyl group, where the guanidinyl group is a suitable amino group that can be converted to a cationic group useful herein.

In another aspect, the polyamine can be a biodegradable synthetic polymer or naturally-occurring polymer. The mechanism by which the polyamine can degrade will vary depending upon the polyamine that is used. In the case of natural polymers, they are biodegradable because there are enzymes that can hydrolyze the polymer chain. For example, proteases can hydrolyze natural proteins like gelatin. In the case of synthetic biodegradable polyamines, they also possess chemically labile bonds. For example, Q-aminoesters have hydrolyzable ester groups.

In one aspect, the polyamine includes a polysaccharide, a protein, peptide, or a synthetic polyamine. Polysaccharides bearing two or more amino groups can be used herein. In one aspect, the polysaccharide is a natural polysaccharide such as chitosan or chemically modified chitosan. Similarly, the protein can be a synthetic or naturally-occurring compound. In another aspect, the polyamine is a synthetic polyamine such as poly(D-aminoesters), polyester amines, poly(disulfide amines), mixed poly(ester and amide amines), and peptide crosslinked polyamines.

In one aspect, the pharmaceutically-acceptable salt of the polyamine can be an amine-modified natural polymer. For example, the amine-modified natural polymer can be gelatin modified with one or more alkylamino groups, heteroaryl groups, or an aromatic group substituted with one or more amino groups. Examples of alkylamino groups are depicted in Formulae IV-VI

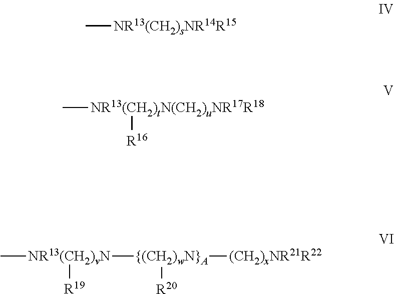

wherein R¹³-R²² are, independently, hydrogen, an alkyl group, or a nitrogen containing substituent;
s, t, u, v, w, and x are an integer from 1 to 10; and
A is an integer from 1 to 50,
where the alkylamino group is covalently attached to the natural polymer. In one aspect, if the natural polymer has a carboxyl group (e.g., acid or ester), the carboxyl group can be reacted with an alkyldiamino compound to produce an amide bond and incorporate the alkylamino group into the polymer. Thus, referring to formulae IV-VI, the amino group NR¹³ is covalently attached to the carbonyl group of the natural polymer.

As shown in formula IV-VI, the number of amino groups can vary. In one aspect, the alkylamino group is
—NHCH₂NH₂, —NHCH₂CH₂NH₂, —NHCH₂CH₂CH₂NH₂, —NHCH₂CH₂CH₂CH₂NH₂,
—NHCH₂CH₂CH₂CH₂CH₂NH₂,
—NHCH₂NHCH₂CH₂CH₂NH₂,
—NHCH₂CH₂NHCH₂CH₂CH₂NH₂,
—NHCH₂CH₂CH₂NHCH₂CH₂CH₂CH₂NHCH₂CH₂CH₂NH₂,
—NHCH₂CH₂NHCH₂CH₂CH₂CH₂NH₂,
—NHCH₂CH₂NHCH₂CH₂CH₂NHCH₂CH₂CH₂NH₂, or
—NHCH₂CH₂NH(CH₂CH₂NH)_{d}CH₂CH₂NH₂, where d is from 0 to 50.

In one aspect, the pharmaceutically-acceptable salt of the amine-modified natural polymer can include an aryl group having one or more amino groups directly or indirectly attached to the aromatic group. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine. In another aspect, the biodegradable polyamine can be gelatin modified with ethylenediamine.

In other aspects, the polycationic salt can be a dendrimer. The dendrimer can be a branched polymer, a multi-armed polymer, a star polymer, and the like. In one aspect, the dendrimer is a polyalkylimine dendrimer, a mixed amino/ether dendrimer, a mixed amino/amide dendrimer, or an amino acid dendrimer. In another aspect, the dendrimer is poly(amidoamine), or PAMAM. In one aspect, the dendrimer has 3 to 20 arms, wherein each arm comprises an amino group.

In one aspect, the polycationic salt includes a polyacrylate having one or more pendant protonated amino groups. For example, the backbone of the polycationic salt can be derived from the polymerization of acrylate monomers including, but not limited to, acrylates, methacrylates, acrylamides, methacrylamides, and the like. In one aspect, the polycationic salt backbone is derived from polyacrylamide. In other aspects, the polycationic salt is a random co-polymer. In other aspects, the polycationic salt is a block copolymer, where segments or portions of the co-polymer possess cationic groups or neutral groups depending upon the selection of the monomers and method used to produce the co-polymer.

In another aspect, the polycationic salt is a pharmaceutically-acceptable salt of a protamine. Protamines are polycationic, arginine-rich proteins that play a role in condensation of chromatin into the sperm head during spermatogenesis. As by-products of the fishing industry, commercially available protamines, purified from fish sperm, are readily available in large quantity and are relatively inexpensive. A non-limiting example of a protamine useful herein is salmine. In another aspect, the protamine is clupein.

In one aspect, the polycationic salts is a polymer with a plurality of guanidinyl groups. In one aspect, the guanidinyl groups are pendant to the polymer backbone. The number of guanidinyl groups present on the polycation ultimately determines the charge density of the polycation. In one aspect, the guanidinyl group can be derived from a residue of arginine attached to a polymer backbone.

The polyguanidinyl polymer can be a homopolymer or copolymer having a plurality of guanidinyl groups. In one aspect, the polyguanidinyl copolymer is a synthetic compound prepared by the free radical polymerization between a monomer such as an acrylate, a methacrylate, an acrylamide, a methacrylamide, or any combination thereof, and a guanidinyl monomer of formula I

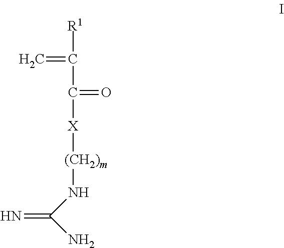

wherein R¹ is a hydrogen or an alkyl group, X is oxygen or NR⁵, where R⁵ is a hydrogen or an alkyl group, and m is from 1 to 10, or the pharmaceutically acceptable salt thereof. In one aspect, when the neutral compound of formula I is used to produce the polymer, the resulting polymer can be subsequently reacted with an acid such as, for example, hydrochloric acid or ammonium chloride, to produce the polycationic salt.

In one aspect, in the compound of formula I, R¹ is methyl, X is NH, and m is 3. In another aspect, the monomer is methacrylamide, methacrylamide, N-(2-hydroxypropyl)methacrylamide (HPMA), N-[3-(N′-dicarboxymethyl)aminopropyl]methacrylamide (DAMA), N-(3-aminopropyl)methacrylamide, N-(1,3-dihydroxypropan-2-yl) methacrylamide, N-isopropylmethacrylamide, N-hydroxyethylacrylamide (HEMA), or any combination thereof.

In a further aspect, the mole ratio of the guanidinyl monomer of formula I to the monomer is from 1:20 to 20:1, or is 1:20, 1:19, 1:18, 1:17, 1:16, 1:15, 1:14, 1:13, 1:12, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1, where any ratio can be a lower and upper end-point of a range (e.g., 2:1 to 5:1, etc.). In one aspect, the mole ratio of the guanidinyl monomer of formula I to the monomer is from 3:1 to 4:1. In another aspect, the polyguanidinyl polymer is a homopolymer derived from the guanidinyl monomer of formula I.

The polyguanidinyl copolymer can be synthesized using polymerization techniques known in the literature such as, for example, RAFT polymerization (i.e., reversible addition-fragmentation chain-transfer polymerization) or other methods such as free radical polymerization. In one aspect, the polymerization reaction can be carried out in an aqueous environment. As discussed above, the polyguanidinyl copolymer can be prepared initially as a neutral polymer followed by treatment with an acid to produce the pharmaceutically-acceptable salt.

In another aspect, multiple copolymers with controlled M_{w} and narrow polydispersity indices (PDIs) can be synthesized by RAFT polymerization. In one aspect, the pharmaceutically-acceptable salt of the polyguanidinyl copolymer has an average molecular weight (M_{w}) from about 1 kDa to about 100 kDa, or can be about 1, 2, 3, 4, 5, 10, 15, 20, 25, 30, 35, 40, 45, 50, 55, 60, 65, 70, 75, 80, 85, 90, 95, or 100 kDa, where any value can be a lower and upper end-point of a range (e.g., 10 to 25 kDa, etc.).

In another aspect, the pharmaceutically-acceptable salt of the polyguanidinyl copolymer is a multimodal polyguanidinyl copolymer. The term “multimodal polyguanidinyl copolymer” is a polyguanidinyl copolymer with a molecular mass distribution curve being the sum of at least two or more molecular mass unimodal distribution curves. In one aspect, the polyguanidinyl copolymer has a multimodal distribution of polyguanidinyl copolymer molecular mass with modes between 5 and 100 kDa, or can be about 5, 10, 15, 20, 25, 30, 35, 40, 45, 50, 55, 60, 65, 70, 75, 80, 85, 90, 95, or 100 kDa, where any value can be a lower and upper end-point of a range (e.g., 10 to 30 kDa, etc.).

In another aspect, the number of guanidinyl side groups in the pharmaceutically-acceptable salt of the polyguanidinyl copolymer can vary from about 10 to about 100 mol % of the. total polymer sidechains, or can be about 10, 15, 20, 25, 30, 35, 40, 45, 50, 55, 60, 65, 70, 75, 80, 85, 90, 95, or 100 mol %, where any value can be a lower and upper end-point of a range (e.g., 60 to 90 mol %, etc.). In one aspect, the guanidinyl side groups are from about 70 to about 80 mol % of the polyguanidinyl copolymer. Conversely, comonomer concentration can vary from about 50 to about 0 mol %, or can be about 50, 45, 40, 35, 30, 25, 20, 15, 10, 5, or 0 mol %, where any value can be a lower and upper end-point of a range (e.g., 10 to 40 mol %, etc.). In one aspect, the Mn, PDI, and structures of the copolymers can be verified by size exclusion chromatography (SEC), ¹H NMR, and ¹³C NMR or other common techniques. Exemplary procedures for preparing and characterizing copolymers useful herein are provided in the Examples below.

The concentration of the of the polycationic salt in the injectable compositions described herein can vary depending upon the application of the composition. In one aspect, the concentration of the of the polycationic salt used to produce the injectable compositions described herein is from 100 mg/mL to 1,000 mg/mL, or 100 mg/mL, 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, 650 mg/mL, 700 mg/mL, 750 mg/mL, 800 mg/mL, 850 mg/mL, 900 mg/mL, 950 mg/mL, 1,000 mg/mL, where any value can be a lower and upper end-point of a range (e.g., 200 mg/mL to 500 mg/mL, etc.).

### Polyanionic Salts

The polyanionic salt is a compound with a plurality of anionic groups and pharmaceutically-acceptable cationic counterions, where there is a 1:1 stoichiometric ratio of the anionic groups to cationic counterions.

In one aspect, the polyanionic polyelectrolyte is derived by dissolving a polyanionic salt in water. In one aspect, the polyanionic salts described herein can be produced by adjusting the pH of a solution of a compound with a plurality of acidic groups (e.g., carboxylic acid groups) with the addition of a base to produce the corresponding anionic groups. In various aspects, bases which may be employed to form pharmaceutically acceptable polyanionic salts include alkali metal hydroxides, carbonates, acetate, etc. In one aspect, once the polyanion has been prepared, excess ions can be removed from the polyanion by filtration or dialysis prior to drying (e.g., lyophilization) to produce the polyanionic salt with stoichiometric amounts of cationic counterions relative to the number of anionic groups.

In one aspect, the cationic counterions of the polyanionic salt are monovalent cations such as, for example, sodium, potassium or ammonium ions. In another aspect, the counterions of the polyanionic salt are multivalent ion such as, for example, calcium, magnesium ions, or mixtures thereof.

In one aspect, the polyanionic salt is composed of a polymer backbone with a plurality of anionic groups and pharmaceutically-acceptable cationic counterions. The anionic groups can be pendant to the polymer backbone and/or incorporated within the polymer backbone. In certain aspects, (e.g., biomedical applications), the polyanionic salt is any biocompatible polymer possessing anionic groups.

In one aspect, the polyanionic salt can be a pharmaceutically-acceptable salt of a synthetic polymer or naturally-occurring polymer. Examples of naturally-occurring polyanions include glycosaminoglycans such as chondroitin sulfate, heparin, heparin sulfate, dermatan sulfate, keratin sulfate, and hyaluronic acid. In other aspects, proteins having a net negative charge at neutral pH or proteins with a low pl can be used as naturally-occurring polyanions described herein. The anionic groups can be pendant to the polymer backbone and/or incorporated in the polymer backbone.

When the polyanionic salt is a synthetic polymer, it is generally any polymer possessing anionic groups or groups that can be ionized to anionic groups. Examples of groups that can be converted to anionic groups include, but are not limited to, carboxylate, sulfonate, boronate, sulfate, borate, phosphonate, or phosphate.

In one aspect, the polyanionic salt is a polyphosphate. In another aspect, the polyanionic salt is a polyphosphate compound having from 5 to 90 mole % phosphate groups. In another aspect, the polyanionic salt has from 10 to 1,000 phosphate groups, or 10, 50, 100, 150, 200, 250, 300, 350, 400, 450, 500, 550, 600, 650, 700, 750, 800, 850, 900, 950, or 1,000 phosphate groups, where any value can be a lower and upper end-point of a range (e.g., 100 to 300, etc.).

In one aspect, the polyphosphate can be a naturally-occurring compound such as, for example, DNA, RNA, or highly phosphorylated proteins like phosvitin (an egg protein), dentin (a natural tooth phosphoprotein), casein (a phosphorylated milk protein), or bone proteins (e.g. osteopontin).

In another aspect, the polyanionic salt can be a synthetic polypeptide made by polymerizing the amino acid serine and then chemically or enzymatically phosphorylating the polypeptide. In another aspect, the polyanionic salt can be produced by the polymerization of phosphoserine. In one aspect, the polyphosphate can be produced by chemically or enzymatically phosphorylating a protein (e.g., natural serine- or threonine-rich proteins). In a further aspect, the polyphosphate can be produced by chemically phosphorylating a polyalcohol including, but not limited to, polysaccharides such as cellulose or dextran. The polyanionic polymers can subsequently be converted to pharmaceutically-acceptable salts.

In another aspect, the polyphosphate can be a synthetic compound. For example, the polyphosphate can be a polymer with pendant phosphate groups attached to the polymer backbone and/or present in the polymer backbone. (e.g., a phosphodiester backbone).

In one aspect, the polyanionic salt includes a polyacrylate having one or more pendant phosphate groups. For example, the polyanionic salt can be derived from the polymerization of acrylate monomers including, but not limited to, acrylates, methacrylates, acrylamides, methacrylamides, and the like. In other aspects, the polyanionic salt is a block co-polymer, where segments or portions of the co-polymer possess anionic groups and neutral groups depending upon the selection of the monomers used to produce the co-polymer. In one aspect, the anionic group can be a plurality of carboxylate, sulfate, sulfonate, borate, boronate, phosphonate, or phosphate groups.

In one aspect, the polyanionic salt is a polymer having a plurality of fragments of formula XI

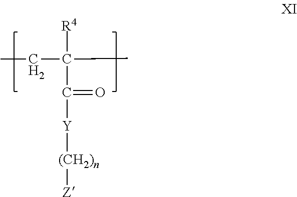

wherein R⁴ is hydrogen or an alkyl group;
n is from 1 to 10;
Y is oxygen, sulfur, or NR³⁰, wherein R³⁰ is hydrogen, an alkyl group, or an aryl group;
Z′ is a pharmaceutically-acceptable salt of an anionic group.

In one aspect, Z′ in formula XI is carboxylate, sulfate, sulfonate, borate, boronate, a substituted or unsubstituted phosphate, or a phosphonate. In another aspect, Z′ in formula XI is sulfate, sulfonate, borate, boronate, a substituted or unsubstituted phosphate, or a phosphonate, and n in formulae XI is 2.

In one aspect, the polyanionic salt can be an inorganic polyphosphate including a cyclic inorganic polyphosphate having the formula (PₙO₃ₙ)ⁿ⁻, a linear inorganic polyphosphate having the formula (PnO₃Oₙ₊₁)ⁿ⁺2−, or a combination thereof. In one aspect, the polyanionic salt is an inorganic polyphosphate possessing a plurality of phosphate groups (e.g., NaPO₃)ₙ, where n is 10 to 1,000 or 10, 50, 100, 150, 200, 250, 300, 350, 400, 450, 500, 550, 600, 650, 700, 750, 800, 850, 900, 950, or 1,000 phosphate groups, where any value can be a lower and upper end-point of a range (e.g., 100 to 300, etc.). Examples of inorganic phosphates include, but are not limited to, Graham salts, hexametaphosphate salts, and triphosphate salts. The counterions of these salts can be monovalent cations such as, for example, Na⁺, K⁺, NH₄⁺, or a combination thereof. In one aspect, the polyanionic salt is sodium hexametaphosphate.

In another aspect, the polyanionic salt is an organic polyphosphate. In one aspect, polymers with phosphodiester backbones connecting organic moieties (e.g., DNA or synthetic phosphodiesters) are organic polyphosphates useful herein.

In another aspect, the polyanionic salt is a pharmaceutically-acceptable salt of a phosphorylated sugar. The sugar can be a hexose or pentose sugar. Additionally, the sugar can be partially or fully phosphorylated. In one aspect, the phosphorylated sugar is inositol hexaphosphate (IP6).

The concentration of the of the polyanionic salt in the injectable compositions described herein can vary depending upon the application of the composition. In one aspect, the concentration of the of the polyanionic salt used to produce the injectable compositions described herein is from 100 mg/mL to 1,000 mg/mL, or 100 mg/mL, 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, 650 mg/mL, 700 mg/mL, 750 mg/mL, 800 mg/mL, 850 mg/mL, 900 mg/mL, 950 mg/mL, 1,000 mg/mL, where any value can be a lower and upper end-point of a range (e.g., 200 mg/mL to 500 mg/mL, etc.).

### Contrast Agents

The injectable compositions described herein can include one or more contrast agents that permit the visualization of the solid formed in situ after the injectable composition has been administered to the subject.

In one aspect, the contrast agent is a radiographic contrast agent. Further in this aspect, the radiographic contrast agent can be tantalum metal particles (Ta), gold particles, or an iodide salt (e.g., sodium iodide). In one aspect, up to 30% (w/w) of Ta can be included in the formulations. In one aspect, inclusion of Ta can be beneficial to interventional radiologists in the operating room. In another aspect, the contrast agent can be a fluoroscopic contrast agent. Further in this aspect, the fluoroscopic contrast agent can be tantalum oxide (TaO₂, Ta₂O₅) particles. In one aspect, the contrast agent can be tantalum particles having a particle size from 0.5 μm to 50 μm, 1 μm to 25 μm, 1 μm to 10 μm, or 1 μm to 5 μm. In another aspect, contrast agent is tantalum particles in the amount of 10% to 60%, 20% to 50%, or 20% to 40%.

The injectable compositions described herein include one or more transient contrast agents, where the contrast agent readily diffuses out of the solid formed upon administration to the subject, providing temporary contrast.

In one aspect, the transient contrast agent is a non-ionic compound. In another aspect, the transient contrast agent is water-soluble. In one aspect, the transient contrast agent is an iodinated organic compound, where one or more iodine atoms are covalently bonded to the organic compound. Iodinated organic contrast agents are a class of iodine-containing organic compounds. This set of compounds are derivatives of 2,3,5-triidobenzoic acid to produce different commercially available compounds, such as iopamidol, iodixanol, iohexol, iopromide, iobtiridol, iomeprol, iopentol, iopamiron, ioxilan, iotrolan, iotrol and ioversol, iopanoate, diatrizoic acid, iothalamate, and ioxaglate, various side chains are added to the parent compound. These sidechains modify the solubility, toxicity, and osmolality of the compound. Iodixanol is a dimer of the parent compound, producing a molecule with 6 iodine atoms. Structures for these compounds and the parent compound 2, 3, 5-triidobenzoic acid are shown in FIG. 2. In another aspect, the iodinated organic compound is an iodinated oil such as, for example, ethiodized poppyseed oil (Lipiodol).

The concentration of the transient contrast agent in the injectable compositions can vary depending upon the application. In one aspect, the concentration of the transient contrast agent in the injectable composition is from 10 mgl/mL to 1,000 mgl/mL, or is 10 mgl/mL, 25 mgl/mL, 50 mgl/mL, 75 mgl/mL, 100 mgl/mL, 125 mgl/mL, 150 mgl/mL, 175 mgl/mL, 200 mgl/mL, 225 mgl/mL, 250 mgl/mL, 275 mgl/mL, 300 mgl/mL, 325 mgl/mL, 350 mgl/mL, 375 mgl/mL, 400 mgl/mL, 425 mgl/mL, 450 mgl/mL, 475 mgl/mL, 500 mgl/mL, 525 mgl/mL, 550 mgl/mL, 575 mgl/mL, 600 mgl/mL, 625 mgl/mL, 650 mgl/mL, 675 mgl/mL, 700 mgl/mL, 725 mgl/mL, 750 mgl/mL, 775 mgl/mL, 800 mgl/mL, 825 mgl/mL, 850 mgl/mL, 875 mgl/mL, 900 mgl/mL, 925 mgl/mL, 950 mgl/mL, 975 mgl/mL, or 1,000 mgl/mL, 100 mgl/mL, 100 mgl/mL, 100 mgl/mL, 100 mgl/mL, 100 mgl/mL, 100 mgl/mL, where any value can be a lower and upper end-point of a range (e.g., 400 mgl/mL to 600 mgl/mL, etc.).

In one aspect, the majority of the transient contrast agent that diffuses from the solid formed in situ is such that the transient contrast agent cannot be detected by imaging techniques such as, for example, fluoroscopy or CT. In one aspect, up to 70%, up to 80%, up to 90%, up to 95%, or up to 100% of the transient contrast agent diffuses out of the solid from 5 minutes to 48 hours once the solid is produced in situ, or 5 minutes, 10 minutes, 30 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 36 hours, 42 hours, or 48 hours, 2 days, 5 days, 10 days, 15 days, 20 days, 25 days, or 30 days, where any value can be a lower and upper end-point of a range (e.g., 1 hour to 3 hours, etc.).

When the injectable compositions described include a transient contrast agent, they possess numerous advantages over previous established embolics. The transient contrast agents readily diffuse from the solid produced in situ upon administration to the subject. The transient contrast agents permit facile imaging of the solid produced in situ at the time of administration of the injectable composition; however, the majority if not all of the transient contrast agent diffuses from the solid over a period of time. In contrast to other embolic agents with immediate or short-term radiopacity, where the agent diminishes in seconds after administration to a subject, the transient contrast agent in the solid produced by the injectable compositions described herein remain in the solid for a period of hours. In other words, there is contrast of an intermediate duration between rapidly dissipating contrast agents and permanent contrast agents; release of the transient contrast agent from the solid is delayed over an extended period of time. This feature permits the delivered embolic to remain visible throughout the duration of the embolization procedure, which results in better confirmation of material placement as well as provide guidance for subsequent injections during the procedure. This temporary radiopacity or contrast provides utility in that it does not interfere in any subsequent imaging, including fluoroscopy or CT, or future treatment of nearby targets. It also allows electrocautery to be used without sparking, in contrast to liquid embolization agents with metallic contrast. Thus, the injectable compositions described herein thus address the shortcomings regarding the use of permanent contrast agents.

### Reinforcing Component

In another aspect, the injectable compositions described herein also include a reinforcing component. The term “reinforcing component” is defined herein as any component that enhances or modifies one or more mechanical or physical properties of the solids produced herein (e.g., cohesiveness, fracture toughness, elastic modulus, dimensional stability after curing, color, visibility etc.). The mode in which the reinforcing component can enhance the mechanical properties of the solid can vary and will depend on the selection of the components used to prepare the injectable composition and reinforcing component. Examples of reinforcing component useful herein are provided below.

In one aspect, the reinforcing component is a coil or fiber. In a further aspect, the coil or fiber can be platinum, plastic, nylon, another natural or synthetic fiber, a polymerizable monomer, a nanostructure, a micelle, a liposome, a water-insoluble filler, or any combination thereof. In one aspect, the coil or fiber is administered concurrently with the injectable composition. In another aspect, the coil or fiber is administered sequentially either before or after the injectable composition.

In other aspects, the reinforcing component can be a water-insoluble filler. The filler can have a variety of different sizes and shapes, ranging from particles (micro and nano) to fibrous materials. The selection of the filler can vary depending upon the application of the injectable composition.

The fillers useful herein can be composed of organic and/or inorganic materials. In one aspect, the nanostructures can be composed of organic materials like carbon or inorganic materials including, but not limited to, boron, molybdenum, tungsten, silicon, titanium, copper, bismuth, tungsten carbide, aluminum oxide, titanium dioxide, molybdenum disulphide, silicon carbide, titanium diboride, boron nitride, dysprosium oxide, iron (III) oxide-hydroxide, iron oxide, manganese oxide, titanium dioxide, boron carbide, aluminum nitride, or any combination thereof.

In one aspect, the filler comprises a metal oxide, a ceramic particle, or a water insoluble inorganic salt. Examples of fillers useful herein include those manufactured by SkySpring Nanomaterials, Inc., which is listed below.

### Metals and Non-Metal Elements

Ag, 99.95%, 100 nm
Ag, 99.95%, 20-30 nm
Ag, 99.95%, 20-30 nm, PVP coated
Ag, 99.9%, 50-60 nm
Ag, 99.99%, 30-50 nm, oleic acid coated
Ag, 99.99%, 15 nm, 10 wt %, self-dispersible
Ag, 99.99%, 15 nm, 25 wt %, self-dispersible
Al, 99.9%, 18 nm
Al, 99.9%, 40-60 nm
Al, 99.9%, 60-80 nm
Al, 99.9%, 40-60 nm, low oxygen
Au, 99.9%, 100 nm
Au, 99.99%, 15 nm, 10 wt %, self-dispersible
B, 99.9999%
B, 99.999%
B, 99.99%
B, 99.9%
B, 99.9%, 80 nm
Diamond, 95%, 3-4 nm
Diamond, 93%, 3-4 nm
Diamond, 55-75%, 4-15 nm
Graphite, 93%, 3-4 nm
Super Activated Carbon, 100 nm
Co, 99.8%, 25-30 nm
Cr, 99.9%, 60-80 nm
Cu, 99.5%, 300 nm
Cu, 99.5%, 500 nm
Cu, 99.9%, 25 nm
Cu, 99.9%, 40-60 nm
Cu, 99.9%, 60-80 nm
Cu, 5-7 nm, dispersion, oil soluble
Fe, 99.9%, 20 nm
Fe, 99.9%, 40-60 nm
Fe, 99.9%, 60-80 nm
Carbonyl-Fe, micro-sized
Mo, 99.9%, 60-80 nm
Mo, 99.9%, 0.5-0.8 □m
Ni, 99.9%, 500 nm (adjustable)
Ni, 99.9%, 20 nm
Ni coated with carbon, 99.9%, 20 nm
Ni, 99.9%, 40-60 nm
Ni, 99.9%, 60-80 nm
Carbonyl-Ni, 2-3 □m
Carbonyl-Ni, 4-7 □m
Carbonyl-Ni-AI (Ni Shell, Al Core)
Carbonyl-Ni-Fe Alloy
Pt, 99.95%, 5 nm, 10 wt %, self-dispersible
Si, Cubic, 99%, 50 nm
Si, Polycrystalline, 99.99995%, lumps
Sn, 99.9%, <100 nm
Ta, 99.9%, 60-80 nm
Ti, 99.9%, 40-60 nm
Ti, 99.9%, 60-80 nm
W, 99.9%, 40-60 nm
W, 99.9%, 80-100 nm
Zn, 99.9%, 40-60 nm
Zn, 99.9%, 80-100 nm

### Metal Oxides

AlOOH, 10-20 nm, 99.99%
Al₂O₃ alpha, 98+%, 40 nm
Al₂O₃ alpha, 99.999%, 0.5-10 □m
Al₂O₃ alpha, 99.99%, 50 nm
Al₂O₃ alpha, 99.99%, 0.3-0.8 □m
Al₂O₃ alpha, 99.99%, 0.8-1.5 □m
Al₂O₃ alpha, 99.99%, 1.5-3.5 □m
Al₂O₃ alpha, 99.99%, 3.5-15 □m
Al₂O₃ gamma, 99.9%, 5 nm
Al₂O₃ gamma, 99.99%, 20 nm
Al₂O₃ gamma, 99.99%, 0.4-1.5 □m
Al₂O₃ gamma, 99.99%, 3-10 □m
Al₂O₃ gamma, Extrudate
Al₂O₃ gamma, Extrudate
Al(OH)₃, 99.99%, 30-100 nm
Al(OH)₃, 99.99%, 2-10 □m
Aluminium Iso-Propoxide (AIP), C₉H₂₁O₃Al, 99.9%
AlN, 99%, 40 nm
BaTiO₃, 99.9%, 100 nm
BBr₃, 99.9%
B₂O₃, 99.5%, 80 nm
BN, 99.99%, 3-4 □m
BN, 99.9%, 3-4 □m
B₄C, 99%, 50 nm
Bi₂O₃, 99.9%, <200 nm
CaCO₃, 97.5%, 15-40 nm
CaCO₃, 15-40 nm
Ca₃(PO₄)₂, 20-40 nm
Ca₁₀(PO₄)₆(OH)₂, 98.5%, 40 nm
CeO₂, 99.9%, 10-30 nm
CoO, <100 nm
Co₂O₃, <100 nm
Co₃O₄, 50 nm
CuO, 99+%, 40 nm
Er₂O₃, 99.9%, 40-50 nm
Fe₂O₃ alpha, 99%, 20-40 nm
Fe₂O₃ gamma, 99%, 20-40 nm
Fe₃O₄, 98+%, 20-30 nm
Fe₃O₄, 98+%, 10-20 nm
Gd₂O₃, 99.9%<100 nm
HfO₂, 99.9%, 100 nm
In₂O₃:SnO₂=90:10, 20-70 nm
In₂O₃, 99.99%, 20-70 nm
In(OH)₃, 99.99%, 20-70 nm
LaB₆, 99.0%, 50-80 nm
La₂O₃, 99.99%, 100 nm
LiFePO₄, 40 nm
MgO, 99.9%, 10-30 nm
MgO, 99%, 20 nm
MgO, 99.9%, 10-30 nm
Mg(OH)₂, 99.8%, 50 nm
Mn₂O₃, 98+%, 40-60 nm
MoCl₅, 99.0%
Nd₂O₃, 99.9%, <100 nm
NiO, <100 nm
Ni₂O₃, <100 nm
Sb₂O₃, 99.9%, 150 nm
SiO₂, 99.9%, 20-60 nm
SiO₂, 99%, 10-30 nm, treated with Silane Coupling Agents
SiO₂, 99%, 10-30 nm, treated with Hexamethyldisilazane
SiO₂, 99%, 10-30 nm, treated with Titanium Ester
SiO₂, 99%, 10-30 nm, treated with Silanes
SiO₂, 10-20 nm, modified with amino group, dispersible
SiO₂, 10-20 nm, modified with epoxy group, dispersible
SiO₂, 10-20 nm, modified with double bond, dispersible
SiO₂, 10-20 nm, surface modified with double layer, dispersible
SiO₂, 10-20 nm, surface modified, super-hydrophobic & oleophilic, dispersible
SiO₂, 99.8%, 5-15 nm, surface modified, hydrophobic & oleophilic, dispersible
SiO₂, 99.8%, 10-25 nm, surface modified, super-hydrophobic, dispersible
SiC, beta, 99%, 40 nm
SiC, beta, whisker, 99.9%
Si₃N₄, amorphous, 99%, 20 nm
Si₃N₄ alpha, 97.5-99%, fiber, 100 nmλ800 nm
SnO₂, 99.9%, 50-70 nm
ATO, SnO₂:Sb₂O₃=90:10, 40 nm
TiO₂ anatase, 99.5%, 5-10 nm
TiO₂ Rutile, 99.5%, 10-30 nm
TiO₂ Rutile, 99%, 20-40 nm, coated with SiO₂, highly hydrophobic
TiO₂ Rutile, 99%, 20-40 nm, coated with SiO₂/Al₂O₃
TiO₂ Rutile, 99%, 20-40 nm, coated with Al₂O₃, hydrophilic
TiO₂ Rutile, 99%, 20-40 nm, coated with SiO₂/Al₂O₃/Stearic Acid
TiO₂ Rutile, 99%, 20-40 nm, coated with Silicone Oil, hydrophobic
TiC, 99%, 40 nm
TiN, 97+%, 20 nm
WO₃, 99.5%, <100 nm
WS₂, 99.9%, 0.8 μm
WCl₅, 99.0%
Y₂O₃, 99.995%, 30-50 nm
ZnO, 99.8%, 10-30 nm
ZnO, 99%, 10-30 nm, treated with silane coupling agents
ZnO, 99%, 10-30 nm, treated with stearic acid
ZnO, 99%, 10-30 nm, treated with silicone oil
ZnO, 99.8%, 200 nm
ZrO₂, 99.9%, 100 nm
ZrO₂, 99.9%, 20-30 nm
ZrO₂-3Y, 99.9%, 0.3-0.5 □m
ZrO₂-3Y, 25 nm
ZrO₂-5Y, 20-30 nm
ZrO₂-8Y, 99.9%, 0.3-0.5 □m
ZrO₂-8Y, 20 nm
ZrC, 97+%, 60 nm In one aspect, the filler is nanosilica. Nanosilica is commercially available from multiple sources in a broad size range. For example, aqueous Nexsil colloidal silica is available in diameters from 6-85 nm from Nyacol Nanotechnologies, Inc. Amino-modified nanosilica is also commercially available, from Sigma Aldrich for example, but in a narrower range of diameters than unmodified silica.

In another aspect, the filler can be composed of calcium phosphate. In one aspect, the filler can be hydroxyapatite, which has the formula Ca₅(PO₄)₃OH. In another aspect, the filler can be a substituted hydroxyapatite. A substituted hydroxyapatite is hydroxyapatite with one or more atoms substituted with another atom. The substituted hydroxyapatite is depicted by the formula M₅X₃Y, where M is Ca, Mg, Na; X is PO₄ or CO₃; and Y is OH, F, Cl, or CO₃. Minor impurities in the hydroxyapatite structure may also be present from the following ions: Zn, Sr, Al, Pb, Ba. In another aspect, the calcium phosphate comprises a calcium orthophosphate. Examples of calcium orthophosphates include, but are not limited to, monocalcium phosphate anhydrate, monocalcium phosphate monohydrate, dicalcium phosphate dihydrate, dicalcium phosphate anhydrous, octacalcium phosphate, beta tricalcium phosphate, alpha tricalcium phosphate, super alpha tricalcium phosphate, tetracalcium phosphate, amorphous tricalcium phosphate, or any combination thereof. In other aspects, the calcium phosphate can also include calcium-deficient hydroxyapatite, which can preferentially adsorb bone matrix proteins.

In certain aspects, the filler can be functionalized with one or more amino or activated ester groups. In this aspect, the filler can be covalently attached to the polycation or polyanion. For example, aminated silica can be reacted with the polyanion possessing activated ester groups to form new covalent bonds.

### Bioactive Agents

The injectable compositions described herein can include one or more bioactive agents. In one aspect, the bioactive agent is an antibiotic, a pain reliever, an immune modulator, a growth factor, an enzyme inhibitor, a hormone, a messenger molecule, a cell signaling molecule, a receptor agonist, an oncolytic virus, a chemotherapy agent, an anti-angiogenic agent, a receptor antagonist, a nucleic acid, or any combination thereof.

In one aspect, the bioactive agent can be a nucleic acid. The nucleic acid can be an oligonucleotide, deoxyribonucleic acid (DNA), ribonucleic acid (RNA) including mRNA, or peptide nucleic acid (PNA). The nucleic acid of interest can be a nucleic acid from any source, such as a nucleic acid obtained from cells in which it occurs in nature, recombinantly produced nucleic acid, or chemically synthesized nucleic acid, or chemically modified nucleic acids. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In other aspects, the bioactive agent is used in bone treatment applications. For example, the bioactive agent can be bone morphogenetic proteins (BMPs) and prostaglandins. When the bioactive agent is used to treat osteoporosis, bioactive agents known in the art such as, for example, bisphonates, can be delivered locally to the subject by the injectable compositions and solids produced therefrom.

In certain aspects, the filler used to produce the injectable composition can also possess bioactive properties. For example, when the filler is a silver particle, the particle can also behave as an anti-microbial agent. The rate of release can be controlled by the selection of the materials used to prepare the injectable composition, as well as the charge of the bioactive agent if the agent has ionizable groups. Thus, in this aspect, the solid produced from the injectable composition can perform as a localized controlled drug release depot. It may be possible to simultaneously fix tissue and bones as well as deliver bioactive agents to provide greater patient comfort, accelerate bone healing, and/or prevent infections.

In one aspect, the bioactive agent is an FDA-approved anti-angiogenic agent. In one aspect, the anti-angiogenic agent is a tyrosine kinase inhibitor (TKI). Not wishing to be bound by theory, angiogenesis is, in large part, initiated and maintained by cell signaling through receptor tyrosine kinases (RTKs). In one aspect, RTKs include receptors for several angiogenesis promoters, including VEGF, which stimulates vascular permeability, proliferation, and migration of endothelial cells; PDGF, which recruits pericytes and smooth muscle cells that support the budding endothelium; and FGF, which stimulates proliferation of endothelial cells, smooth muscle cells, and fibroblasts. In one aspect, the anti-angiogenic agent is a TKI such as sunitinib malate (SUN), pazopanib hydrochloride (PAZ), sorafenib tosylate (SOR), vandetanib (VAN), cabozantinib, or any combination thereof.

In another aspect, the bioactive agent can be humanized anti-VEGF and anti-VEGFR Fab′ fragments. In this aspect, electrostatic interactions can control release kinetics. In one aspect, the native charge of the Fab′ fragment is sufficient to interact with the polyelectrolyte components in the injectable composition. In another aspect, the native charge of the Fab′ fragment is insufficient to interact with the polyelectrolyte components in the injectable composition and the Fab′ fragment is modified to increase charge density by attaching a short polyelectrolyte to reactive sulfhydryl groups using maleamide conjugation chemistries.

In one aspect, the anti-angiogenic agent is an anti-VEGF antibody. In a still further aspect, the anti-VEGF antibody is bevacizumab or is a biosimilar anti-VEGF antibody, or is an anti-VEGF antibody derivative such as, for example, ranibizumab.

### Kits

Described herein are kits for making the injectable compositions. In one aspect, the kit includes (a) a composition comprising a mixture of at least one polycationic salt and at least one polyanionic salt, and (b) instructions for making the injectable composition. In another aspect, the kit includes (a) at least one polycationic salt, (b) at least one polyanionic salt, and (c) instructions for making the injectable composition.

The polycationic salt and polyanionic salt used herein can be stored as dry powders for extended periods of time. In one aspect, the kit can include dry powders of the polycationic salt and polyanionic salt as separate components in separate vials, or a mixture of the polycationic salt and polyanionic salt as a dry powder or solid in a single container. In other aspects, the kit can include aqueous solutions of the polycationic salt and polyanionic salt as separate components (e.g., in separate vials) or a mixture of the polycationic salt and polyanionic salt in water.

The kits also include instructions for making the injectable compositions. As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can include one or multiple documents and are meant to include future updates.

The kits can also include additional components as described herein (e.g., reinforcing components, bioactive agents, etc.). In other aspects, the kits can include optional mechanical components such as, for example, syringes, microcatheters, and other devices for mixing and delivering the injectable compositions to a subject.

### Preparation of the Injectable Compositions

The preparation of the injectable compositions described herein can be performed using a number of techniques and procedures. Exemplary techniques for producing the injectable compositions are provided in the Examples. In one aspect, a powder composed of a mixture of the at least one polycationic salt and the at least one polyanionic salt are mixed with a contrast agent in water for a sufficient time to produce an injectable composition.

In one aspect, one or more additional agents (e.g., contrast agents, reinforcing agent or bioactive agent) can be added after the injectable composition has been formed. In another aspect, the anti-angiogenic agent and the one or more additional agents (e.g., reinforcing agent or bioactive agent) can be added during the formation of the injectable composition.

In one aspect, the pH of the injectable composition is from 6 to 9, 6.5 to 8.5, 7 to 8, or 7 to 7.5. In another aspect, the pH of the composition is 7.2, which is the normal physiological pH in blood.

The injectable compositions described herein are stable solutions (i.e., a liquid composition of polyelectrolytes with no distinguishable separation into distinct phases). Although the components used to produce the injectable composition can be used in dry powder form then subsequently mixed with water, the injectable compositions can be formulated as water-borne formulations and stored for future use. In certain aspects, one or more additional salts can be added to the injectable composition to prevent association of the polycationic polyelectrolytes and the polyanionic polyelectrolytes in the injectable composition. In one aspect, the salt is a monovalent salt. For example, sodium chloride can be added to the injectable composition to produce a stable composition as defined herein. The concentration of the monovalent salt can vary depending upon the molecular weight, concentration, and charge ratio of the polycationic and polyanionic salts. In other aspects, additional monovalent salt is not needed to produce the injectable compositions as stable solutions.

Depending upon the application site in the subject and delivery device dimensions, the viscosity of the of the injectable composition can be modified accordingly. This is an important feature with respect to medical applications such as, for example, transarterial microcatheter delivery, where different size microcatheters are needed for different applications. For example, modifying the concentration and/or molecular weight of the polycationic salt and/or the polyanionic salt can be used to modify the viscosity of the injectable composition.

In one aspect, the injectable composition has a viscosity of from 10 cp to 20,000 cp, or 10 cp, 25 cp, 50 cp, 75 cp, 100 cp, 125 cp, 150 cp, 200 cp, 225 cp, 250 cp, 275 cp, 300 cp, 325 cp, 350 cp, 375 cp, 400 cp, 425 cp, 450 cp, 475 cp, 500 cp, 1,000 cp, 1,500 cp, 2,000 cp, 2,500 cp, 3,000 cp, 3,500 cp, 4,000 cp, 4,500 cp, 5,000 cp, 5,500 cp, 6,000 cp, 6,500 cp, 7,000 cp, 7,500 cp, 8,000 cp, 8,500 cp, 9,000 cp, 9,500 cp, 10,000 cp, 11,000 cp, 12,000 cp, 13,000 cp, 14,000 cp, 15,000 cp, 10,000 cp, 16,000 cp, 17,000 cp, 18,000 cp, 19,000 cp, or 20,000 cp, where any value can be a lower and upper end-point of a range (e.g., 1,500 cp to 7,000 cp, etc.).

### Applications of the Injectable Compositions

The injectable compositions described herein have numerous benefits and biomedical applications. As discussed above, the injectable compositions are fluids that are readily injectable via a narrow-gauge device, catheter, needle, cannula, or tubing. The injectable compositions are water-borne eliminating the need for potentially toxic solvents.

The injectable compositions described herein are fluids at ion concentrations higher than the ion concentration of the application site in the subject, but insoluble solids at the ion concentration of the application site. When the injectable compositions are introduced into a subject at a lower ion concentration relative to the ion concentration of the injectable composition, the composition forms a porous solid in situ at the application site as the ion concentration in the injectable composition approaches the application site ion concentration. The solid that is subsequently produced has higher mechanical moduli than those of the initial fluid form of the injectable composition.

In one aspect, the injectable solution is delivered as pulses such that solid particles are periodically formed and released from the tip of the catheter within the subject. The in situ formed solid particles can be carried by the bloodstream to a distal location from the catheter tip to create a synthetic embolus.

In one aspect, the ion concentration of the injectable composition is the sum of the cationic and anionic counterions present in the composition. In another aspect, the ion concentration of the injectable composition is the sum of the cationic and anionic counterions present in the composition as well as additional ions that are added to the composition (e.g., the addition of NaCl to the composition). In one aspect, the composition has an ion concentration that is about 1.5 to about 20 times greater than the ion concentration in the subject, or about 1.5, 2, 3, 4, 5, 6, 7, 8, 9, 10, 11, 12, 13, 14, 15, 16, 17, 18, 19, or 20 times greater than the ion concentration in the subject, where any value can be a lower and upper end-point of a range (e.g., 2 times to 15 times). In another aspect, the ionic concentration in the composition is from 0.5 M to 2.0 M, or 0.5 M, 0.75 M, 1.0 M, 1.25 M, 1.5 M, 1.75 M, or 2.0 M, where any value can be a lower and upper end-point of a range (e.g., 0.75 M to 1.5 M).

The injectable compositions can form solids in situ under physiological conditions. The physiological sodium and chloride concentration is approximately 150 mM. Thus, when injectable compositions having an ion concentration greater than 150 mM are introduced to a subject (e.g., injected into a mammal), the injectable composition is converted to a porous solid at the site of application. Thus, the injectable compositions described herein have numerous medical and biological applications, which are described in detail below.

In another aspect, the injectable compositions described herein can be used to fill a void in a subject. In certain conditions, it is desirable to fill a void in a subject to prevent or avoid significant health risks. The void as defined herein as an empty space within a bone, muscle, skin, cartilage, tissue, or organ in the subject. In the alternative, the void can be a pouch or sac attached to a bone, muscle, skin, cartilage, tissue, or organ in the subject. In one aspect, the void is filled with a fluid. For example, the void is a seroma, which is a fluid-filled void in a tissue often occurring after surgery.

In one aspect, the injectable composition can be delivered into the void with the use of a catheter or needle. In one aspect, the void can be completely filled by the injectable composition. In another aspect, the void can be partially filled (i.e., less than 100%) by the injectable composition.

In one aspect, the void is a left atrial appendage (LAA). A left atrial appendage (LAA) is a small pouch found in the top left of the heart (the left atrium). The LAA can facilitate the development of blood clots in people with atrial fibrillation (~6 million), putting them at a higher risk of stroke. These patients can be treated with medications, with surgery, or with an interventional procedure.

There are two common LAA closure devices, the Watchman (Boston Scientific) and the Amplatzer Amulet (Abbott). Both use a trans-septal approach, piercing the septum of the right and left atrium to allow the passage of a large 12-14 Fr catheter which delivers a closure device that anchors at and seals off the opening of the LAA.

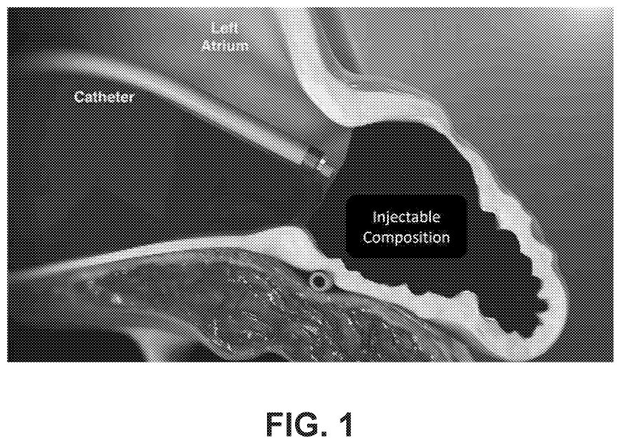

The injectable compositions described herein can be delivered through catheter or needle without the need to pierce the atrial septum. The injectable composition could be delivered by catheter through the left ventricle to the left atrium (FIG. 1) and will fill and close the left atrial appendage. The solid produced in situ fills the available space within the LAA and solidifies over the course of several minutes. As it solidifies, the material is sterically held in place by the ridges of the LAA topography.

In another aspect, the injectable compositions described herein can fill a void in a lymph node is addition to reducing or inhibiting flow of lymph from the right lymphatic duct, the thoracic duct, or both. Here, the injectable composition can be delivered into the lymph to reduce or prevent the flow of lymph to the lymph ducts.

In other aspects, the injectable compositions described herein can encapsulate, scaffold, seal, or hold one or more bioactive agents. Thus, the injectable compositions can be used as a delivery device or implantable drug depot.

As discussed above, the injectable compositions can be used as synthetic embolic agents. However, in other aspects, the injectable composition described herein can include one or more additional embolic agents. Embolic agents commercially-available are microparticles used for embolization of blood vessels. The size and shape of the microparticles can vary. In one aspect, the microparticles can be composed of polymeric materials. An example of this is Bearin™ nsPVA particles manufactured by Merit Medical Systems, Inc., which are composed of polyvinyl alcohol ranging in size from 45 μm to 1,180 μm. In another aspect, the embolic agent can be a microsphere composed of a polymeric material. Examples of such embolic agents include Embosphere® Microspheres, which are made from trisacryl cross-linked gelatin ranging in size from 40 μm to 1,200 μm; HepaSphere™ Microspheres (spherical, hydrophilic microspheres made from vinyl acetate and methyl acrylate) ranging in size from 30 μm to 200 μm; and QuadraSphere® Microspheres (spherical, hydrophilic microspheres made from vinyl acetate and methyl acrylate) ranging in size from 30 μm to 200 μm, all of which are manufactured by Merit Medical Systems, Inc. In another aspect, the microsphere can be impregnated with one or more metals that can be used as a contrast agent. An example of this is EmboGold® Microspheres manufactured by Merit Medical Systems, Inc., which are made from cross-linked trisacryl gelatin impregnated with 2% elemental gold ranging in size from 40 μm to 1,200 μm.

In another aspect, the injectable compositions described herein can be used in combination with one or more mechanical vascular devices such as, for example, embolic coils, fibers, and the like. In one aspect, the mechanical embolic is first administered to a blood vessel in the subject using techniques known in the art followed by the administration of the injectable composition to the blood vessel within or in close proximity to the mechanical device.

It is also contemplated that the solids produced from the injectable compositions described herein can encapsulate, scaffold, seal, or hold one or more bioactive agents. Thus, the solid can be used as a delivery device or implantable drug depot.

### ASPECTS

Aspect 1. A method for filling a void in a subject comprising introducing into the void a composition comprising water, one or more polycationic polyelectrolytes and anionic counterions, and one or more one polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in the subject, whereupon introduction of the composition into the subject a solid is produced in situ in the void of the subject.
Aspect 2. The method of Aspect 1, wherein the void is within a bone, muscle, skin, cartilage, tissue, or organ or the void is a pouch or sac attached to a bone, muscle, skin, cartilage, tissue, or organ in the subject.
Aspect 3. The method of Aspect 1, wherein the void is a left atrial appendage.
Aspect 4. The method of Aspect 1, wherein the void is in a lymph node.
Aspect 5. The method of any one of Aspects 1-4, wherein the composition is introduced into the void by a catheter or needle.
Aspect 6. The method of any one of Aspects 1-5, wherein the polycationic polyelectrolyte is derived by dissolving a polycationic salt in water.
Aspect 7. The method of any one of Aspects 1-5, wherein the polycationic polyelectrolyte is derived from a polycationic hydrochloride salt in water.
Aspect 8. The method of any one of Aspects 1-7, wherein the polycationic salt comprises a pharmaceutically-acceptable salt of a polyamine.
Aspect 9. The method of Aspect 8, wherein the polyamine comprises two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.
Aspect 10. The method of Aspect 8 or 9, wherein the pharmaceutically-acceptable salt of the polyamine comprises a dendrimer having 3 to 20 arms, wherein each arm comprises a terminal amino group.
Aspect 11. The method of any one of Aspects 1-7, wherein the polycationic salt comprises a polyacrylate comprising two or more pendant amino groups, wherein the amino group comprises a primary amino group, a secondary amino group, tertiary amino group, a quaternary amine, an alkylamino group, a heteroaryl group, a guanidinyl group, an imidazolyl, or an aromatic group substituted with one or more amino groups.
Aspect 12. The method of any one of Aspects 1-7, wherein the polycationic salt comprises a pharmaceutically-acceptable salt of a biodegradable polyamine.
Aspect 13. The method of Aspect 12, wherein the pharmaceutically-acceptable salt of the biodegradable polyamine comprises a polysaccharide, a protein, a peptide, a recombinant protein, a synthetic polyamine, a protamine, a branched polyamine, or an amine-modified natural polymer.
Aspect 14. The method of Aspect 12, wherein the pharmaceutically-acceptable salt of the biodegradable polyamine comprises gelatin modified with an alkyldiamino compound.
Aspect 15. The method of any one of Aspects 1-7, wherein the polycationic salt comprises a pharmaceutically-acceptable salt of a protamine.
Aspect 16. The method of any one of Aspects 1-7, wherein the polycationic salt is a pharmaceutically-acceptable salt of salmine or clupein.
Aspect 17. The method of any one of Aspects 1-7, wherein the polycationic salt is a pharmaceutically-acceptable salt of natural polymer or a synthetic polymer containing two or more guanidinyl sidechains.
Aspect 18. The method of any one of Aspects 1-7, wherein the polycationic salt comprises a pharmaceutically-acceptable salt of a polyacrylate comprising two or more pendant guanidinyl groups.
Aspect 19. The method of any one of Aspects 1-7, wherein the polycationic salt comprises a pharmaceutically-acceptable salt of a homopolymer comprising pendant guanidinyl groups.
Aspect 20. The method of any one of Aspects 1-7, wherein the polycationic salt comprises a pharmaceutically-acceptable salt of a copolymer comprising two or more pendant guanidinyl groups.
Aspect 21. The method of any one of Aspects 1-7, wherein the polycationic salt comprises a pharmaceutically-acceptable salt of a synthetic polyguanidinyl copolymer comprising an acrylate, methacrylate, acrylamide, or methacrylamide backbone and two or more guanidinyl groups pendant to the backbone.
Aspect 22. The method of any one of Aspects 1-7, wherein the polycationic salt comprises a pharmaceutically-acceptable salt of a synthetic polyguanidinyl copolymer comprising the polymerization product between a monomer selected from the group consisting of an acrylate, a methacrylate, an acrylamide, a methacrylamide, or any combination thereof and a pharmaceutically-acceptable salt of compound of formula I

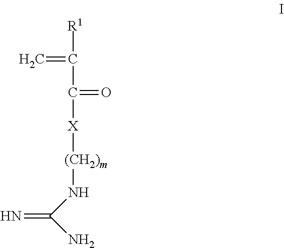

wherein R¹ is hydrogen or an alkyl group, X is oxygen or NR⁵, where R⁵ is hydrogen or an alkyl group, and m is from 1 to 10.
Aspect 23. The method of Aspect 22, wherein the polycationic salt comprises a copolymerization product between the compound of formula I and an acrylate, a methacrylate, an acrylamide, or a methacrylamide,
Aspect 24. The method of Aspect 22, wherein the polycationic salt comprises a copolymerization product between the compound of formula I and methacrylamide, N-(2-hydroxypropyl)methacrylamide (HPMA), N-[3-(N′-dicarboxymethyl)aminopropyl]methacrylamide (DAMA), N-(3-aminopropyl)methacrylamide, N-(1,3-dihydroxypropan-2-yl) methacrylamide, N-isopropylmethacrylamide, N-hydroxyethylacrylamide (HEMA), or any combination thereof.
Aspect 25. The method of Aspect 22, wherein Riis methyl, X is NH, m is 3.
Aspect 26. The method of Aspect 22, wherein the mole ratio of the guanidinyl monomer of formula I to the comonomer is from 1:20 to 20:1.
Aspect 27. The method of Aspect 22, wherein the polyguanidinyl copolymer has an average molar mass from 1 kDa to 1,000 kDa.
Aspect 28. The method of any one of Aspects 1-27, wherein the polyanionic polyelectrolyte is derived by dissolving a polyanionic salt in water.
Aspect 29. The method of Aspect 28, wherein the polyanionic salt comprises a pharmaceutically-acceptable salt of a synthetic polymer or a naturally-occurring polymer.
Aspect 30. The method of Aspect 28 or 29, wherein the polyanionic salt comprises two or more carboxylate, sulfate, sulfonate, borate, boronate, phosphonate, or phosphate groups.
Aspect 31. The method of any one of Aspects 28-30, wherein the polyanionic salt comprises a pharmaceutically-acceptable salt of a glycosaminoglycan or an acidic protein.
Aspect 32. The method of Aspect 31, wherein the glycosaminoglycan comprises chondroitin sulfate, heparin, heparin sulfate, dermatan sulfate, keratin sulfate, or hyaluronic acid.
Aspect 33. The method of Aspect 28, wherein the polyanionic salt comprises a pharmaceutically-acceptable salt of a protein having a net negative charge at a pH of 6 or greater.
Aspect 34. The method of Aspect 28, wherein the polyanionic salt comprises a pharmaceutically-acceptable salt of a polymer comprising anionic groups pendant to the backbone of the polymer, incorporated in the backbone of the polymer backbone, or a combination thereof.
Aspect 35. The method of Aspect 28, wherein the polyanionic salt comprises a pharmaceutically-acceptable salt of a homopolymer or copolymer comprising two or more anionic groups.
Aspect 36. The method of Aspect 28, wherein the polyanionic salt is a copolymer comprising two or more fragments having the formula XI

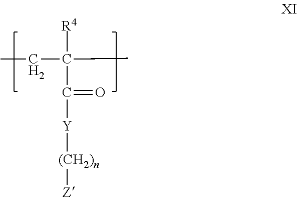

wherein R⁴ is hydrogen or an alkyl group;
n is from 1 to 10;
Y is oxygen, sulfur, or NR³⁰, wherein R³⁰ is hydrogen, an alkyl group, or an aryl group;
Z′ is a pharmaceutically-acceptable salt of an anionic group.
Aspect 37. The method of Aspect 36, wherein Z′ is carboxylate, sulfate, sulfonate, borate, boronate, a substituted or unsubstituted phosphate or phosphonate.
Aspect 38. The method of Aspect 37, wherein n is 2.
Aspect 39. The method of any one of Aspects 28-30, wherein the polyanionic salt comprises a polyphosphate.
Aspect 40. The method of Aspect 39, wherein the polyphosphate comprises a natural polymer or a synthetic polymer.
Aspect 41. The method of Aspect 39, wherein the polyphosphate comprises polyphosphoserine.
Aspect 42. The method of Aspect 39, wherein the polyphosphate comprises a polyacrylate comprising two or more pendant phosphate groups.
Aspect 43. The method of Aspect 39, wherein the polyphosphate is the copolymerization product between a phosphate acrylate and/or phosphate methacrylate with one or more additional polymerizable monomers.
Aspect 44. The method of any one of Aspects 28-30, wherein the polyanionic salt has from 10 to 1,000 phosphate groups.
Aspect 45. The method of any one of Aspects 28-30, wherein the polyanionic salt comprises a pharmaceutically-acceptable salt of an inorganic polyphosphate, an organic polyphosphate, or a phosphorylated sugar.
Aspect 46. The method of Aspect 45, wherein the polyanionic salt comprises a pharmaceutically-acceptable salt of inositol hexaphosphate.
Aspect 47. The method of Aspect 45, wherein the polyanionic salt comprises a hexametaphosphate salt.
Aspect 48. The method of Aspect 45, wherein the polyanionic salt comprises sodium hexametaphosphate.
Aspect 49. The method of any one of Aspects 28-30, wherein the polyanionic salt comprises a pharmaceutically-acceptable salt of cyclic inorganic polyphosphate, a linear inorganic polyphosphate, or a combination thereof.
Aspect 50. The method of any one of Aspects 28-30, wherein the polyanionic salt comprises a pharmaceutically-acceptable salt of a polyacrylate comprising two or more pendant phosphate groups.
Aspect 51. The method of any one of Aspects 28-30, wherein the polyanionic salt comprises a pharmaceutically-acceptable salt of the copolymerization product between a phosphate or phosphonate acrylate or phosphate or phosphonate methacrylate with one or more additional polymerizable monomers.
Aspect 52. The method of any one of Aspects 1-51, wherein the composition further comprises a contrast agent.
Aspect 53. The method of Aspect 52, wherein the contrast agent is a radiographic contrast agent.
Aspect 54. The method of Aspect 52, wherein the contrast agent is tantalum metal particles, gold particles, or tantalum oxide particles.
Aspect 55. The method of Aspect 52, wherein the contrast agent is a transient contrast agent.
Aspect 56. The method of Aspect 55, wherein the transient contrast agent comprises an iodinated organic compound.
Aspect 57. The method of Aspect 56, wherein the iodinated organic compound comprises iopamidol, iodixanol, iohexol, iopromide, iobtiridol, iomeprol, iopentol, iopamiron, ioxilan, iotrolan, iotrol and ioversol, iopanoate, diatrizoic acid, iothalamate, ioxaglate, or any combination thereof.
Aspect 58. The method of Aspect 56, wherein the iodinated organic compound comprises an iodinated oil.
Aspect 59. The method of any one of Aspects 55-58, wherein the concentration of the transient contrast agent in the composition is from 50 mgl/mL to 450 mgl/mL.
Aspect 60. The method of any one of Aspects 55-59, wherein the transient contrast agent diffuses out of the solid and becomes undetectable in 5 minutes to 30 days.
Aspect 61. The method of any one of Aspects 1-60, wherein the composition further comprises a reinforcing component, wherein the reinforcing component comprises natural or synthetic fibers, water-insoluble filler particles, a nanoparticle, or a microparticle.
Aspect 62. The method of Aspect 61, wherein the reinforcing component comprises natural or synthetic fibers, water-insoluble filler particles, a nanoparticle, or a microparticle.
Aspect 63. The method of any one of Aspects 1-62, wherein the composition further comprises one or more bioactive agents, wherein the bioactive agent comprises an antibiotic, a pain reliever, an immune modulator, a growth factor, an enzyme inhibitor, a hormone, a messenger molecule, a cell signaling molecule, a receptor agonist, an oncolytic virus, a chemotherapy agent, a receptor antagonist, a nucleic acid, a chemically-modified nucleic acid, or any combination thereof.
Aspect 64. The method of any one of Aspects 1-63, wherein the composition has a viscosity of from 10 cp to 20,000 cp.
Aspect 65. The method of any one of Aspects 1-64, wherein the total positive/negative charge ratio of the polycationic polyelectrolytes to the polyanionic polyelectrolytes is from 4 to 0.25 and the ion concentration in the composition is from 0.5 M to 2.0 M.
Aspect 66. The method of any one of Aspects 1-65, wherein the concentration of the polycationic polyelectrolytes and the polyanionic polyelectrolytes is sufficient to yield a charge ratio of polycationic polyelectrolytes to polyanionic polyelectrolytes from 0.5:1 to 2:1.
Aspect 67. The method of any one of Aspects 1-66, wherein the composition has a pH of 6 to 9.

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed:

1. A method for filling a void in a subject comprising introducing into the void a composition comprising water, one or more polycationic polyelectrolytes and anionic counterions, and one or more one polyanionic polyelectrolytes and cationic counterions, wherein the composition has an ion concentration that is (i) sufficient to prevent association of the polycationic polyelectrolytes and the polyanionic polyelectrolytes in water and (ii) greater than the concentration of ions in the subject, whereupon introduction of the composition into the subject a solid is produced in situ in the void of the subject.

2. The method of claim 1, wherein the void is within a bone, muscle, skin, cartilage, tissue, or organ or the void is a pouch or sac attached to a bone, muscle, skin, cartilage, tissue, or organ in the subject.

3. The method of claim 1, wherein the void is a left atrial appendage.

4. The method of claim 1, wherein the void is in a lymph node.

5. The method of claim 1, wherein the composition is introduced into the void by a catheter or needle.

6. The method of claim 1, wherein the total positive/negative charge ratio of the polycationic polyelectrolytes to the polyanionic polyelectrolytes is from 4 to 0.25 and the ion concentration in the composition is from 0.5 M to 2.0 M.

7. The method of claim 1, wherein the concentration of the polycationic polyelectrolytes and the polyanionic polyelectrolytes is sufficient to yield a charge ratio of polycationic polyelectrolytes to polyanionic polyelectrolytes from 0.5:1 to 2:1.

8. The method of claim 1, wherein the polycationic polyelectrolyte is derived from a polycationic hydrochloride salt in water.

9. The method of claim 1, wherein the polycationic polyelectrolyte is derived by dissolving a polycationic salt in water.

10. The method of claim 9, wherein the polycationic salt comprises a pharmaceutically-acceptable salt of a polyamine.

11. The method of claim 9, wherein the polycationic salt comprises a pharmaceutically-acceptable salt of a protamine, salmine, or clupein.

12. The method of claim 9, wherein the polycationic salt is a pharmaceutically-acceptable salt of natural polymer or a synthetic polymer containing two or more guanidinyl sidechains.

13. The method of claim 9, wherein the polycationic salt comprises a pharmaceutically-acceptable salt of a polyacrylate comprising two or more pendant guanidinyl groups.

14. The method of claim 9, wherein the polycationic salt comprises a pharmaceutically-acceptable salt of a synthetic polyguanidinyl copolymer comprising an acrylate, methacrylate, acrylamide, or methacrylamide backbone and two or more guanidinyl groups pendant to the backbone.

15. The method of claim 9, wherein the polycationic salt comprises a pharmaceutically-acceptable salt of a synthetic polyguanidinyl copolymer comprising the polymerization product between a monomer selected from the group consisting of an acrylate, a methacrylate, an acrylamide, a methacrylamide, or any combination thereof and a pharmaceutically-acceptable salt of compound of formula I 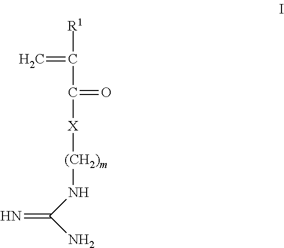
wherein R¹ is hydrogen or an alkyl group, X is oxygen or NR⁵, where R⁵ is hydrogen or an alkyl group, and m is from 1 to 10.

16. The method of claim 1, wherein the polyanionic polyelectrolyte is derived by dissolving a polyanionic salt in water.

17. The method of claim 16, wherein the polyanionic salt comprises a pharmaceutically-acceptable salt of an inorganic polyphosphate, an organic polyphosphate, or a phosphorylated sugar.

18. The method of claim 16, wherein the polyanionic salt comprises a hexametaphosphate salt.

19. The method of claim 16, wherein the polyanionic salt comprises sodium hexametaphosphate.

20. The method of claim 1, wherein the composition further comprises a contrast agent, a reinforcing component, a bioactive agent, or any combination thereof.
